(19) United States
(12) Patent Application Publication
(10) Pub. No.: US 2026/0185926 A1
(43) Pub. Date: Jul. 2, 2026

(54) STARK EFFECT POLARIZATION SPECTROSCOPY AND METHODS

(71) Applicant: Physical Sciences, Inc., Andover, MA (US)

(72) Inventor: Brian E. Brumfield, Concord, NH (US)

(21) Appl. No.: 19/551,077

(22) Filed: Feb. 26, 2026

Related U.S. Application Data
(63) Continuation-in-part of application No. 19/083,589, filed on Mar. 19, 2025.
(60) Provisional application No. 63/679,680, filed on Aug. 6, 2024.

(51) Int. Cl. G01N 21/21; G01N 21/17; G01N 33/00

(52) U.S. Cl. CPC G01N 21/21; G01N 21/1717; G01N 33/0027; G01N 2021/1721; G01N 2201/127

(57) ABSTRACT

A beam such as a laser beam a known polarization state is passed through the container. An electrical field is created in the container to reduce optical interference while preserving the ability to more accurately measure the concentration of one or more gasses in the container. A change in the polarization state of the beam after passage through the container is detected. Based on the detected change in the polarization state of the beam, the presence of the one or more gasses can be determined.

### RELATED APPLICATIONS

This application is a continuation-in-part of U.S. application Ser. No. 19/083,589 filed Mar. 19, 2025 and this application and that application claim benefit of and priority to U.S. Provisional Application Ser. No. 63/679,680 filed Aug. 6, 2024, under 35 U.S.C. §§ 119, 120, 363, 365, and 37 C.F.R. § 1.55 and § 1.78, which is incorporated herein by this reference.

### GOVERNMENT RIGHTS

This invention was made with U.S. Government support under Contract No. 2R44GM135957-02 awarded by the National Institutes of Health. Additionally, this invention was made with U.S. Government support under Contract No. 1R43GM135957-01 awarded by the National Institutes of Health. The Government may have certain rights in the subject invention.

### FIELD OF THE INVENTION

This invention relates to the use of a Stark polarization spectroscopy instrument in the pharmaceutical and other industries.

### BACKGROUND OF THE INVENTION

Vaporized hydrogen peroxide (VHP) is used during the decontamination procedures of pharmaceutical drug production equipment, including barrier isolators, vacuum freeze dryers and other aseptic manufacturing equipment. While VHP is an effective decontaminant, residual VHP after aeration can reduce the efficacy of some drug products due to oxidation. Recent studies have demonstrated that even a residual 30 ppbv concentration of VHP after aeration is sufficient to degrade biologic and other pharmaceuticals resulting in reduced efficacy of the final product, potential harm to patients due to a lack of drug efficacy, and a loss of revenue.

Electrochemical sensors are currently the most widely used detection instrument in the pharmaceutical industry for monitoring VHP concentrations from ˜1 ppmv to 1000 ppmv. These sensors are inexpensive (<$1000 USD), but do not reach the ppbv detection limits needed to protect biologic drugs from oxidization following packaging.

Optical interference from water is a significant challenge when attempting to measure ppbv concentrations of VHP when using spectroscopic measurement techniques during pharmaceutical manufacturing equipment decontamination. Water will always be present during the decontamination process because VHP is generated by evaporating a high concentration (e.g. 35% w/w hydrogen peroxide/water) aqueous solution, and the concentration of water can be many orders larger than VHP. Even the spectral wings of water lines will overlap with the much smaller VHP absorbance. Two approaches are utilized to solve this problem: 1) use of a spectral fitting algorithm to account for and remove water interference, and 2) reduce the pressure of the gas sample to spectrally narrow the absorption lineshapes to minimize the spectral interference from nearby water lines. While both approaches are viable, they limit the spectral regions that can be targeted by an optical VHP sensor, and they also require significant knowledge of the spectroscopic line parameters for the nearby water transitions.

Sensors based on tunable diode laser absorption spectroscopy (TDLAS) or tunable laser spectroscopy (TDL) have been brought to market that are capable of quantifying <30 ppbv of VHP. One product is based on the technique of near-infrared cavity ringdown spectroscopy (NIR-CRDS), while a second instrument uses swept-tunable IR diode laser spectroscopy (IR TDLAS) and a 36 meter multi-pass cell. Spectral interference from water is mitigated in the NIR-CRDS system through spectral fitting and removal of the water signal. The IR-TDLAS system operates at less than atmospheric pressure to reduce pressure broadening so that the VHP spectral line of interest is nearly isolated from nearby water lines. The NIR-CRDS instrument cannot measure VHP concentrations >100 ppmv, and the IR TDLAS instrument cannot measure VHP concentrations >10 ppmv, both due to signal saturation effects. These upper limits of measurement restrict the use of these instruments to later times in the decontamination cycle and must be combined with electrochemical sensors to monitor VHP throughout the entire decontamination cycle. Another drawback to this instrumentation is cost due to system complexity in the case of the NIR-CRDS and for the IR TDLAS instrumentation the additional cost of an infrared laser, optics, and detectors. For example, the CRDS instrument costs >$100K which has limited its adoption by the pharmaceutical industry.

TDLAS offers the capability of real-time, low-consumables, quantitation of VHP down to ppbv levels. The challenges faced by TDLAS in this application are: 1) the measurement of small absorbance losses associated with ppbv concentrations of VHP, and 2) optical interference with water vapor in the infrared and the near-infrared spectral regions.

The measurement of small absorbances can be aided by increasing the effective pathlength through the gas sample and/or measuring the transitions with the largest absorption cross-sections in the infrared portion of the spectrum. The CRDS technique achieves a long-interaction path through a gas sample using a high-finesse optical cavity. This provides adequate sensitivity to measure ppbv concentrations of VHP using a near-infrared tunable laser, however, it requires a high-speed digital acquisition system to record the decay of light leaking out of the cavity on microsecond timescales. When combined with the required temperature and pressure stability for the high-finesse optical cavity, the resulting CRDS instrument is complex, expensive and it is challenging to maintain instrument calibration because even minor contaminants on the high-reflectivity mirrors alter the effective optical cavity pathlength. TDLAS with a multi-pass optical cavity can also measure ppbv quantities of VHP, however it requires the use of a mid-infrared laser source, optics, and detectors—all at a significantly higher cost than near-infrared lasers and optics. Optimization of TDLAS and CRDS instrumentation for ppbv concentrations limits the highest measurable concentration to 10-100 ppmv.

This is an insufficient upper range of VHP detection to enable use of the instrumentation for the complete decontamination cycle, for example, at the start of decontamination when the VHP concentration is very high. Electrochemical sensors are used in industry to cover VHP concentrations from 1-1000 ppmv to overlap with the capabilities of the TDLAS and CRDS instruments. In addition to limited maximum concentration, TDLAS instrumentation requires calibration with a “zero” gas where an analyte of interest is absent, and also with a calibration gas standard that provides a known absorbance.

Chemical assays, such as horseradish peroxidase, remain the gold standard for quantification of ppbv residual levels of VHP following aeration in the decontamination cycle. Chemical assays, however, do not provide real-time quantification of VHP in the process, require chemical reagents as consumables and require skilled technicians to perform the measurement. Chemical assays are rarely used within manufacturing operations to routinely quantify decontamination cycles and VHP residuals.

Stark-enhanced absorption spectroscopy is another approach that could be applied to mitigate optical interference from water vapor. In Stark-enhanced absorption spectroscopy, a modulated electric field is applied to a gas sample containing polar molecules. Due to the Stark-effect, the absorption line profiles of the polar gases are distorted due to the applied electric field resulting in a reduction in the peak absorbance that scales with the magnitude of the applied electric field. By using a phase-sensitive detector called a lock-in amplifier it is possible to record the modulation of the absorption line profile using a wavelength tunable light source transmitted through the gas sample. According to Patent No. DE4338233C2, incorporated herein by this reference, Stark-enhanced absorption spectroscopy with a modulated electric field enables the removal of parasitic etalons and background absorption from water vapor that normally complicates measurements of trace gases in samples. While Stark-enhanced absorption spectroscopy can mitigate optical interference from water vapor—unlike TDLAS and CRDS—it is less sensitive because the applied electric field only modulates a small portion of the absorption of the optical transition. As a consequence, a Stark-enhanced absorption spectrometer to quantify VHP would need to target the mid-IR spectral region where VHP has a sufficient absorption cross-section to enable detection limits below 1 ppmv. A Stark-enhanced absorption spectrometer operating in the mid-IR spectral region would cost as much as an IR TDLAS system.

### BRIEF SUMMARY OF THE INVENTION

Featured is a sensor apparatus and method of measurement that enables users to sensitively and selectively measure gases of relevance to the pharmaceutical industry practices of aseptic fill-finish packaging of pharmaceuticals and to evaluate the closed container integrity of glass vials maintaining the sterility of active pharmaceutical ingredients. In aseptic fill-finish applications, the proposed sensor apparatus and method of measurement are used to ensure that VHP concentrations are reduced to low ppbv levels before packaging for some drug products. A sufficiently sensitive decontamination process monitoring diagnostic is disclosed that can provide real-time VHP concentration measurements in the manufacturing facility during the decontamination and aeration processes.

Disclosed is an optical diagnostic approach that overcomes the limitations encountered in the prior art by TDLAS, CRDS, and Stark-enhanced absorption spectroscopy to quantify ppbv—1000 ppmv levels of VHP during pharmaceutical manufacturing facility decontamination.

The optical diagnostic relies on the application of a modulated electric field to a gas sample that contains VHP and water. The modulated electric field induces linear dichroism and birefringence in the vicinity of optical transitions belonging to the different chemical constituents in the gas sample, and these effects are measured using polarized laser light and a polarimeter. This new diagnostic is referred to as Stark Polarization Spectroscopy (SPS). SPS differs from a related technique called Stark-enhanced absorption spectroscopy where the change in absorption from optical transitions due to the Stark effect is measured with a wavelength tunable light source. Stark-enhanced absorption spectroscopy has been used in atmospheric science and industrial monitoring applications but has never been used to measure gas phase concentrations of VHP. The application of SPS to the problem of VHP quantification during pharmaceutical decontamination overcomes the previous listed challenges in the prior art and offers the following advantages compared to TDLAS, CRDS, and Stark-enhanced absorption spectroscopy.

The measurement of polarization changes in the laser beam is more sensitive than conventional TDLAS or Stark-enhanced absorption spectroscopy, and this is what enables the technique to utilize a single-pass or simple multi-pass arrangement to match the same sensitivity of an absorption technique such as CRDS. This reduces the complexity of an instrument designed to utilize the SPS approach in comparison to TDLAS or Stark-enhanced absorption spectroscopy approaches that require a high-finesse optical cavity or a high-density multi-pass cell geometry.

The SPS technique can be used to measure H₂O and VHP in the MIR spectral region. The high-sensitivity of the technique can enable, in some embodiments, the use of VIS and NIR diode lasers and optics—reducing the cost compared to mid or mid-IR TDLAS instruments or a mid-IR Stark-enhanced absorption spectrometer.

The response of water vapor to the applied electric field is orders of magnitude smaller than VHP. This enables selective quantification of the VHP Stark polarization signal in the presence of water vapor and mitigates the issue of optical interference from water vapor that is problematic for TDLAS measurement approaches.

While Stark-enhanced absorption spectroscopy can enable selective quantification between VHP and water, it lacks the sensitivity of SPS because Stark-enhanced absorption spectroscopy is measuring the absorption of light and not the polarization changes associated with the Stark Effect. Furthermore, Stark-enhanced absorption spectroscopy is less sensitive than TDLAS because the applied electric field only modulates a small portion of the absorption of the optical transition.

A VHP sensor based on the SPS technique does not typically require consumables for calibration. An instrument based on SPS can be zero calibrated by turning off the electric field. Calibration of the SPS signal generated versus the applied electric field strength can be achieved through simultaneous measurement of the absorption and Stark polarization spectrum from water optical transitions. This means that the water vapor present in the decontamination process can be used to calibrate the polarimeter as needed.

The dynamic concentration measurement range of SPS can be adjusted by controlling the value of the applied electric field. To avoid saturation of the polarimeter at higher VHP concentrations the electric field can be reduced.

The application of SPS is also advantageous in closed container integrity testing (CCIT) applications because the polarization signal from a gas is spatially restricted to the region where the electric field is located. By restricting the electric field to the axis of a glass vial, the polarization state of the laser beam is only altered by the presence of gases within the vial, not gases outside of the vial. Thus, gases that have leaked into the vial (e.g. H₂O, O₂, CO₂) due to a lack of closed container integrity can be sensitively measured with minimal or no interference from gases outside of the vial. TDLAS measuring H₂O, CO₂, and O₂ has been used as a non-destructive CCIT method but requires an inert N₂ gas purge outside the vial to remove signal from these gases present in the ambient atmosphere. The application of SPS to CCIT therefore removes the need for an inert N₂ gas purge, enabling SPS to sensitively quantify the concentrations of H₂O, CO₂, and O₂ that have leaked into the headspace of the vial free of atmospheric interference.

Featured is a method of monitoring the concentration of hydrogen peroxide gas in a system. The method preferably includes driving hydrogen peroxide gas and water vapor sampled from the system to a gas cell. The method also includes passing a beam from a source at a known polarization state through the gas cell and the hydrogen peroxide gas and water vapor therein and creating an electrical field in the gas cell to reduce optical interference from water vapor while preserving the ability to more accurately measure the concentration of hydrogen peroxide gas in the gas cell. A change in the polarization state of the beam is detected after it passes through the hydrogen peroxide gas and water vapor. Based on the detected change in the polarization state of the beam, the concentration of the hydrogen peroxide gas in the gas cell (and system) can be determined.

In one embodiment, the system may include an enclosure and gas sampled is from the enclosure following decontamination. The method may include modulating the strength of the electrical field and/or the light beam wavelength to improve the detection sensitivity of the hydrogen peroxide concentration. Polarization optics between the source and the gas cell can be used to control the polarization state of the light. The polarization optics may be configured to pass a known polarization of light through the gas sample exposed to the electrical field in the gas cell. The method may include a polarimeter downstream of the gas cell for detecting the change in the polarization state of the beam after it passes through the hydrogen peroxide gas and water vapor. The method may further include a calibration sequence which simultaneously measures changes in transmission and beam polarization from water vapor used to calibrate the polarimeter. The gas cell may include spaced electrodes connected across a voltage source for creating the electrical field and there may be a polarimeter which receives the beam after it exits the gas cell. The method may further include delivering an output of the polarimeter to a data acquisition system to directly record the output or to a phase sensitive detector which outputs a signal to the voltage source, light source, or both. The light source may be a laser source (e.g., a visible, near infrared or infrared laser).

In one method, an enclosure is filled with hydrogen peroxide gas to decontaminate the enclosure. The hydrogen peroxide gas and water vapor are driven to a gas cell and a polarized light beam from a light source at a known polarization state is passed through the gas cell and the hydrogen peroxide gas and water vapor therein. An electrical field is created in the gas cell to reduce interference of the water vapor while optically measuring the concentration of the hydrogen peroxide gas in the gas cell. A change in the polarization state of the beam after it passes through the hydrogen peroxide gas and water vapor is detected and based on the detected change in the polarization state of the beam, the concentration of the hydrogen peroxide gas in the gas cell and enclosure is measured. The enclosure is ultimately purged to remove hydrogen peroxide gas therefrom, so it does not oxidize drugs processed in the enclosure reducing their efficacy.

In another aspect, a method of monitoring the level of an individual gas in a sample that is composed of multiple different gases is featured. The method includes passing a light beam from a light source at a known polarization state through the sample in a cell, creating an electrical field in the cell to reduce optical interference between the individual gas and other gases in the sample while preserving the ability to more accurately measure the concentration of the individual gas, detecting a change in the polarization state of the beam after it passes through the gas and vapor, and based on the detected change in the polarization state of the beam, determining the concentration of the individual gas.

The gas may be a decontamination gas. The decontamination gas may be comprised of hydrogen peroxide and water vapor. The method may further include modulating the strength of the electrical field and/or the beam wavelength to improve determination of the gas concentration. The method may further include applying Fast Fourier Transform filtering during detecting a change in the polarization state of the beam to remove optical interferences from parasitic etalon fringes not associated with polarization changes from gases. The method may include using a polarization optics to set the polarization state of the beam. The polarizer may be configured to polarize the beam at an angle of 45 degrees with respect to the orientation of the electrical field. The method may include using a polarimeter for detecting the change in the polarization state of the beam after it passes through the gas. The light source may be a near infrared or infrared laser.

In one version, a method of monitoring the concentration of gases in the headspace of a container includes passing a light beam from a light source at a known polarization state through the container headspace, applying an electric field through the headspace of the container such that only optical signals from gases in the headspace of the container are measured while removing the interference from optical signals generated by those gases present in an environment outside the container, detecting a change in the polarization state of the light beam after it passes through the headspace region of the container, and based on the detected change in the polarization state of the light beam, determining the concentration of individual gases in the headspace of the container. The container may include a pharmaceutical product and the headspace may include gases that have leaked into the container such as O₂, N₂, H₂O, and CO₂.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic view of an SPS system for use in conjunction with a pharmaceutical industry barrier isolator;

FIG. 2 is a block diagram of an SPS system that relies on direct modulation of the electric field;

FIG. 3 is a block diagram of an SPS system that exposes a gas sample to a static electric field and probes the induced sample dichroism by modulation of the laser wavelength;

FIG. 4 is a block diagram of a configuration for an SPS system where both the laser wavelength and the electric field are modulated simultaneously;

FIGS. 5A-5B show a direct absorption spectrum (A), an expanded view (B) of the direct absorption spectrum (A) over the wavenumber region where the VHP Stark Polarization spectrum is located in (C), and Stark effect spectrum (C) of water (˜20,000 ppmv) and VHP (˜30 ppmv) acquired simultaneously by scanning the frequency of a near-infrared diode laser (1.4 μm);

FIG. 6 demonstrates the changes in the Stark effect signal from a single infrared optical transition as a function of electric field strength;

FIGS. 7A-7B demonstrate an approach of Fast Fourier Transform filtering applied to a Stark polarization spectrum of VHP to remove interference from parasitic etalon fringes. FIG. 7A shows the VHP Stark polarization with and without application of the Fast Fourier Transform filter, and FIG. 7B plots the spectral amplitudes in frequency space following application of the Fast Fourier transform to the spectral data in FIG. 7A; and

FIG. 8 is a schematic view of an SPS system for enabling measurement discrimination of the same gas inside of a container from that gas outside of the container. In the pharmaceutical industry this can be used for closed container integrity analysis.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

Disclosed herein is a laser-based system and method that uses Stark Polarization Spectroscopy (SPS) for selective and sensitive quantification of a gas, such as a decontamination gas (e.g., VHP) in the presence of vapor such as water vapor, or to selectively monitor the headspace of sealed containers for ingress of gas (e.g. H₂O, CO₂, and O₂) relevant to CCIT applications. The proposed sensor, in some examples, overcomes the challenges of limited dynamic range and optical interference from water encountered by sensors based on laser absorption spectroscopy for quantification of VHP in the pharmaceutical decontamination cycle.

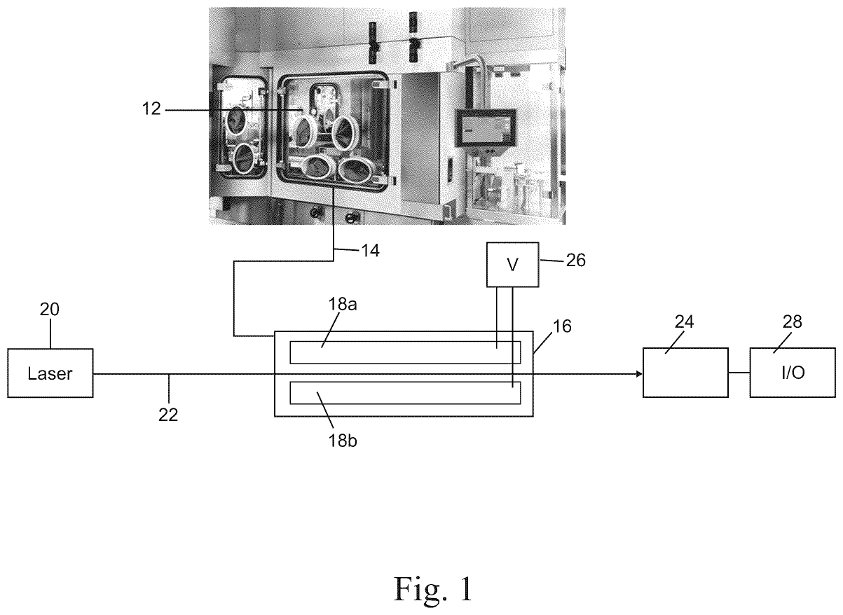

FIG. 1 depicts a system and method in an example where barrier isolator 12 is used to package drug products (e.g., biologic pharmaceutical products). Isolator 12 is sterilized by flooding it with a decontamination gas such as VHP. Then, the VHP is removed from the isolator 12 using an aeration process so the VHP doesn't adversely affect the efficacy of the drug products packaged and processed in isolator 12.

The isolator gas is driven (using a pump, for example) via line 14 to gas cell 16 with spaced interior electrodes 18a, 18b. This can be accomplished at any time during the isolator decontamination process, for example, at the start to ensure a sufficient concentration of VHP is present in the isolator for proper decontamination, during use of the isolator to ensure the VHP concentration is adequate, and also after aeration (purging) of the isolator to ensure the remaining concentration of VHP is below the threshold, which would otherwise adversely affect the efficacy of the drugs processed in the isolator.

A laser subsystem 20 (with or without a polarizer) is oriented to pass a laser beam 22 of a known polarization state through the gas cell 16 (now including VHP) between electrodes 18a, 18b to detection subsystem 24. The polarized light may be linear (plane polarized) or elliptically-polarized, as circular and linearly polarized laser light will interact with the Stark-effect induced linear dichroism and birefringence associated with the VHP and water optical transitions.

A voltage source 26 supplies a voltage across electrodes 18a, 18b (one of which may be held at a ground or reference voltage) to create an electrical field in the gas cell to reduce optical interference from water vapor while preserving the ability to more accurately measure the concentration of the hydrogen peroxide gas in the gas cell.

A detection subsystem 24, including for example, signal processing, computational, computer, and/or other subsystems is configured to detect the change in the polarization state of the laser beam after it passes through the hydrogen peroxide gas and water vapor in the gas cell and, based on the detected change in the polarization state of the laser beam, to determine the concentration of the hydrogen peroxide gas in the gas cell and thus the barrier isolator. The change in polarization state is a function of the concentration of the gas in the gas cell.

The results can be provided to the user or other electronic systems via I/O section 28.

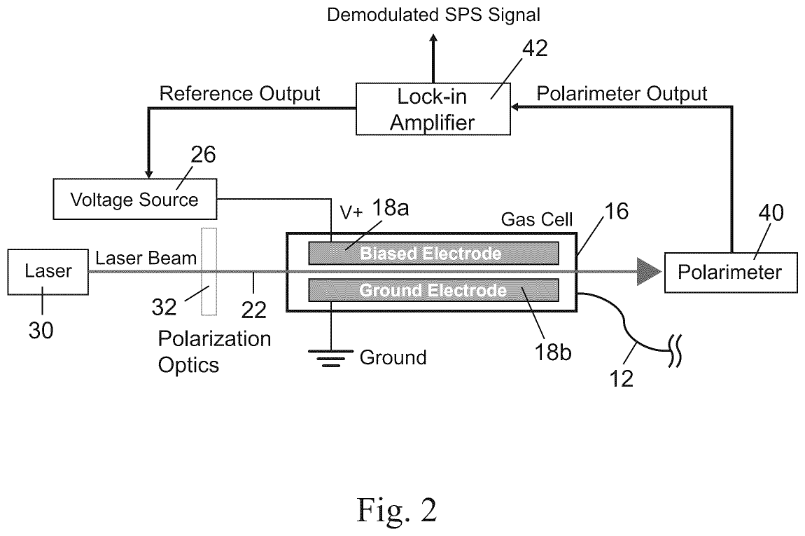

FIGS. 2-4 illustrate exemplary systems where the laser subsystem includes a laser source 30 and polarization optics 32 and the detection subsystem includes a polarimeter 40 and lock-in amplifier 42 as described below. The polarization optics are used to set the polarization state of the light used to probe VHP and water vapor optical transitions in the gas cell. The laser source can be a visible, near-infrared or infrared (e.g., diode) laser.

FIGS. 2, 3, and 4 illustrate three different system preferred approaches to realize an SPS-based sensor for VHP quantification. All three approaches have in common: A wavelength tunable laser source 30, a set of polarization optics 32, a gas cell 16 equipped with two electrodes 18a, 18b, a voltage source 26, a lock-in amplifier, and a polarimeter 40 to measure changes in the polarization state of the laser source after passage through the gas cell to determine gas (e.g. VHP) concentration.

The polarization optics between the laser source and the gas cell are used to set the polarization state of the light from the laser. After the polarization optics, the laser light is coupled into a gas cell and directed through a gap between two electrodes. One electrode inside the gas cell is attached to a voltage source that is used to bias one electrode while the second electrode in the gas cell is connected to a ground potential. When the electrode attached to the voltage source is biased, an electric field exists between the adjacent surfaces of the electrodes. Following passage between the electrodes, the laser beam exits the gas cell and is sent to a polarimeter.

The polarimeter is used to measure changes in the polarization state of the laser light as the laser wavelength is scanned over optical transitions of water and VHP. The polarization state of the laser light is altered due to linear dichroism and birefringence occurring in the vicinity of the optical transitions due to the Quadratic Stark Effect (QSE). It was reasonable to assume optical activity induced in the vicinity of the VHP transitions was due to the Linear Stark Effect. However, through detailed review and study of the Stark polarization signal lineshapes and dependence of the signal amplitudes on the applied electric field it was determined that the observed optical activity induced in the gas medium are solely due to the QSE. To those knowledgeable in the art, there are two different types of the Stark Effect based on the structure of the molecules of interest: The Linear Stark Effect, where the electric field induced changes to rovibrational optical transitions scale to the first power of the electric field vector and the QSE where the electric field induced changes to the rovibrational optical transitions scale with the square of the electric field vector. The Linear Stark Effect only applies to those molecules defined as symmetric top type molecules possessing at least two identical moments of inertia and a permanent electric dipole moment. Advantageously, the QSE applies molecules having three different moments of inertia and does not require the molecule to possess a permanent electric dipole moment. Water and VHP are asymmetric top molecules and are subject to the QSE. In the QSE, the electric field applied to the gas sample induces an orientation-dependent electric dipole moment via the molecular polarizabilities of the water and VHP molecules. Water and VHP molecules in a gas sample experience a torque due to the applied electric field interacting with both the induced electric dipole moment and the permanent electric dipole moment of the molecules. The generated torque on the ensemble of water and VHP molecules results in a partial alignment of the water and VHP molecules in the gas sample. The partial alignment induces anisotropy in the gas phase medium that manifests as differences in the index of refraction (birefringence) and absorption (dichroism) for light polarized parallel and perpendicular to the applied electric field that scales as the square of the applied electric field.

The applied electric field also simultaneously causes a shift in the rovibrational transition frequencies for VHP and water, with the direction of the transition energy shift to either lower or higher energies dictated by the difference in the molecular polarizabilities of the upper and lower states of the molecule. The size of the energy shift is given by: ΔEₜᵣₐₙₛᵢₜᵢₒₙ=α_{eff}E_{field}², where α_{eff} corresponds to the difference in the molecular polarizability between the upper and lower rovibrational states of the optical transition, and is dependent on the rotational and vibrational quantum numbers for the relevant states.

The polarimeter used to measure the changes in the laser beam polarization preferably includes a polarizer and a photodetector unit. The polarimeter can be of a nearly-crossed, 90° type, a balanced polarimeter referred to as a 45° type, or a hybrid-type balanced polarimeter. All three polarimeter types can be configured such that the first polarization optic is a quarter waveplate with the fast axis aligned at 45° with respect to the linear polarization state of light passed through the gas sample exposed to the electric field. To those knowledgeable in the art, this is referred to as the Senarmont configuration, and it is optimized to enhance the detection of weak linear birefringence in gases, liquids, and solids. All three measurement approaches presented in FIGS. 2-4 are compatible with all three polarimeter types. The output voltage from the polarimeter is input into a phase-sensitive detector called a lock-in amplifier to facilitate measurement of the linear dichroic and birefringent signals as the laser wavelength is scanned over water and VHP optical transitions. A lock-in amplifier can also be used to demodulate the time-dependent voltage signal output by the photodetector at the frequency of a reference signal.

In FIGS. 2-4, all three approaches have the lock-in amplifier sending a reference output signal that is used to modulate some aspect of the system resulting in a corresponding modulation of the polarization state of the light, however, a separate external voltage waveform generator can be used that would supply the modulation to the lock-in amplifier and the element that modulates the spectral signal via the shift in the transition energy associated with the QSE. While the current embodiment utilizes a lock-in amplifier to demodulate the encoded Stark polarization signal, it is not required. In an alternative approach the electric field can be held at a constant value while the laser wavelength is swept repetitively over the VHP and water optical transitions. By sweeping the laser wavelength in the kHz regime the Stark polarization spectrum can be recorded and exclude the effects of low-frequency noise commonly referred to as “flicker” noise by those knowledgeable in the art.

In the approach presented in FIG. 2, the reference out from the lock-in amplifier, or external voltage waveform generator, is provided to the voltage source attached to one of the electrodes in the cell.

The voltage source in FIG. 2 is a voltage amplifier, and the reference signal will modulate the electric field at the frequency of the lock-in amplifier resonance. The electric field can be modulated over a wide range of frequencies, however in the current embodiment the electric field modulation was restricted to the kilohertz range due to limitations in the voltage and current output of the voltage amplifier. Because the gas sample is directly modulated in this approach, the demodulated spectrum output by the lock-in amplifier eliminates parasitic optical fringes in the recorded Stark polarization spectrum. In contrast, parasitic etalon fringes limit the long-term averaging stability of sensors based on tunable diode laser absorption spectroscopy.

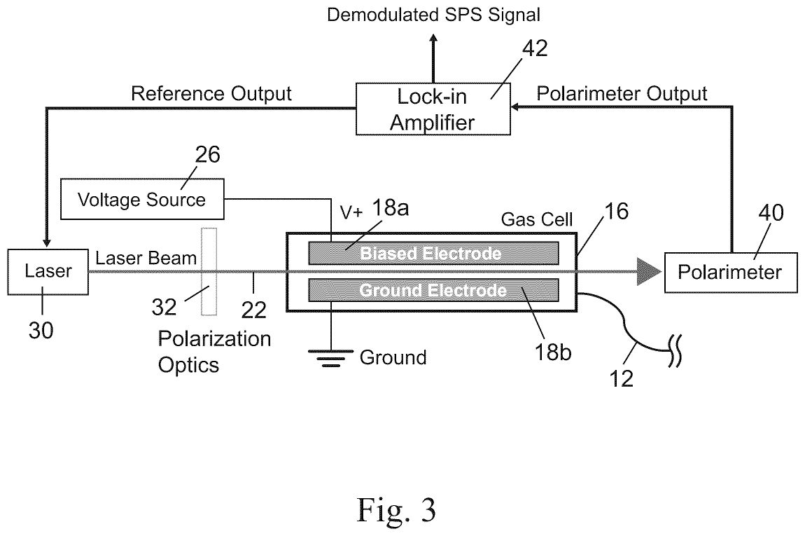

In FIG. 3, the reference signal generated by the lock-in amplifier or an external waveform generator is connected to a current modulation input on the laser driver that results in a modulation of the laser wavelength. In this measurement configuration, the electric field is held at a fixed value as the laser wavelength is scanned over the VHP and water optical transitions. The reference signal from the lock-in amplifier adds an additional modulation of the laser wavelength over a smaller wavelength range and at a higher frequency than the rate at which the laser wavelength is scanned by the laser current or temperature. As the laser wavelength is scanned over an optical transition, the high frequency (>10 kHz) modulation of the laser wavelength is converted into a high-frequency polarization state modulation via interaction with the electric field induced linear dichroism and birefringence. The polarization state modulation is transduced into a voltage modulation by the polarimeter that is then demodulated by the lock-in amplifier at the reference output frequency. Unlike the system arrangement in FIG. 2, this detection method can be vulnerable to parasitic etalon fringes that can reduce the long-term detection sensitivity of the system. The advantage of this approach is that the frequency of wavelength modulation can be higher than the electric field modulation approach due to the limited technical performance of voltage amplifiers. Encoding the Stark polarization spectrum at a higher frequency reduces the short-term measurement noise of the system in comparison to the modulated electric field approach presented in FIG. 2.

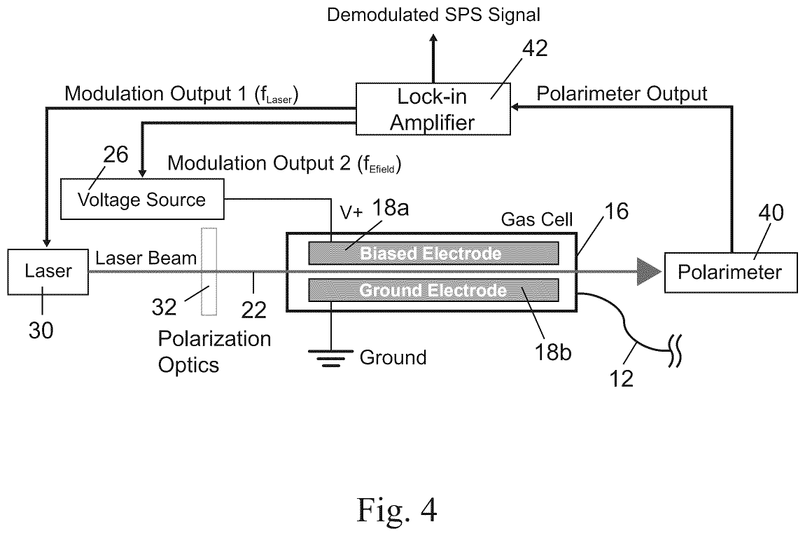

In FIG. 4, the system relies on simultaneous modulation of the laser wavelength (fₗₐₛₑᵣ) and the electric field (f_{Efield}) at two different frequencies. The lock-in amplifier used for this system configuration should be capable of dual demodulation-or two lock-in amplifiers can be used in series to complete demodulation of the signal. The different modulation reference frequencies can come from the lock-in amplifiers or external waveform generators. The system configuration shown in FIG. 4 provides the lowest short term measurement noise by encoding the Stark polarization signal at higher frequencies while the modulation of the electric field enables removal of parasitic etalon fringes. A dual modulation approach has been utilized in Stark Absorption Spectroscopy, but in this measurement configuration only the change in the absorption line shape due to the Stark Effect is measured. The efficacy of a dual modulation approach has been demonstrated to measure the magnetic field induced circular birefringence effects associated with the Faraday Effect for quantification of nitric oxide as described in Dual-Modulation Faraday Rotation Spectroscopy in U.S. Pat. No. 8,947,663B2, 2015 incorporated herein by this reference.

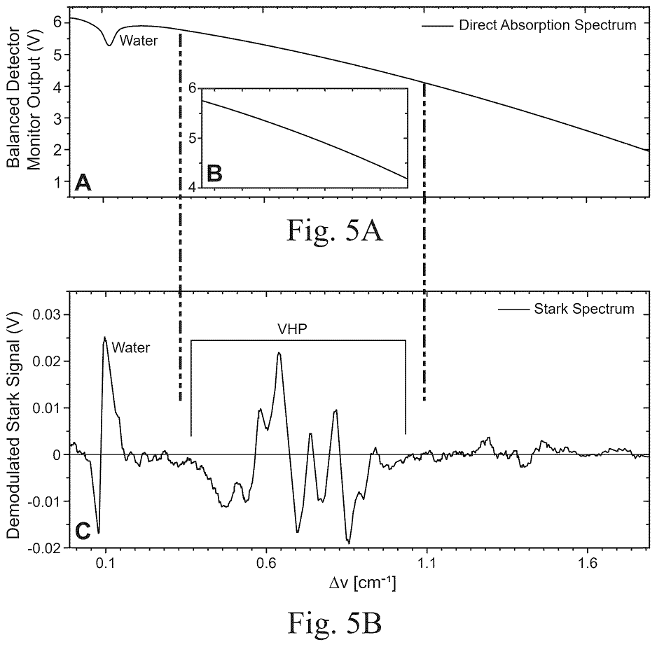

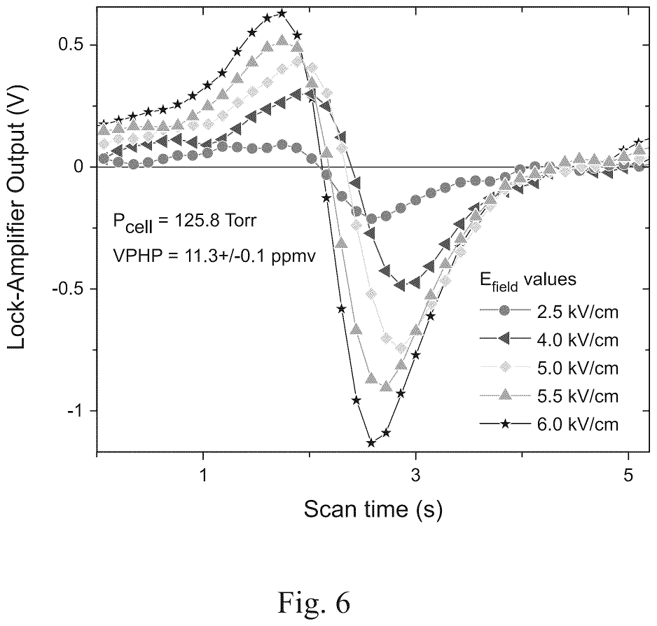

FIG. 5B shows the recorded Stark spectrum (panel C) for VHP and water in the near-infrared using the system configuration presented in FIG. 2 and a balanced polarimeter. In FIG. 5B we observe that the SP signal intensity for the VHP optical transitions is comparable to what is observed for water vapor, while in the simultaneously recorded absorption spectrum shown in FIG. 5A panel (A), a 10% absorption from water vapor is observed with no observable absorption from VHP. The selective enhancement of the spectral signal for VHP in the Stark polarization spectrum is associated with the larger molecular polarizability of VHP compared to water vapor due to differences in the molecular structure. The additional oxygen atom in VHP results in: 1) A larger mean isotropic molecular polarizability because the additional oxygen atom increases the number of electrons in VHP, 2) and a larger rotational anisotropy contribution due to the concentration of electrons along the O—O bond in the VHP molecular structure. The larger molecular polarizability for the VHP transitions results in SP signals comparable to those for water despite the 600 times smaller VHP concentration. The experimental arrangement used in FIG. 6 employed a modulated electric field, and VHP's larger molecular polarizability over water results in bigger shifts in transition energy due to the applied electric field compared to water vapor transitions. The larger shifts in VHP transition energies translate into greater changes in the birefringence and dichroism between the electric field “on” and electric field “off” portions of the electric field modulation cycle that enhance the modulation of the SP signal compared to water. While differences in molecular structure between VHP and water provide a general explanation for the enhancement of the VHP SP signal over water, a complete explanation requires knowledge of the exact vibrational and rotational quantum numbers of the upper and lower states for the VHP optical transitions observed in the SP spectrum in FIG. 6. Currently the rotational and vibrational quantum numbers to describe the upper and lower VHP states are not known.

As shown in FIGS. 5A-5B, by leveraging the larger isotropic and anisotropic polarizability of VHP over water vapor, Stark Polarization Spectroscopy significantly enhances the signal from VHP relative to water, despite the presence of water in the gas sample at concentrations >600 times that of VHP. Gas cell temperature and pressure were 50° C. and 120 Torr respectively, and the peak electric field applied to the sample was 7000 V/cm at a modulation frequency of 5.5 kHz. FIGS. 5A-5B show a direct absorption spectrum (A), an expanded view (B) of the absorption channel (A) where the VHP Stark signals are located in (C), and Stark effect spectrum (C) of water (˜20,000 ppmv) and VHP (˜30 ppmv) acquired simultaneously by scanning the frequency of a near-infrared diode laser (1.4 μm).

This result forms the basis of using SPS to selectively measure VHP and mitigate potential optical interference from more abundant water vapor present in the pharmaceutical manufacturing facility decontamination process. A second attribute of SPS is the signal scales with the magnitude of the applied electric field.

The influence of the electric field value of the SPS signal is illustrated in FIG. 6, where the SPS signal from VHP was recorded with an infrared laser using the type of system configuration shown in FIG. 2 and a 90° type polarimeter. FIG. 6 shows that the scaling of the SPS signal from VHP is proportional to the square of the applied electric field strength. This attribute of SPS is beneficial when the VHP concentration is high (>100 ppmv) early in the pharmaceutical decontamination process. At high concentrations with a fixed electric field amplitude, it is possible to exceed the voltage output of a polarimeter. This refers to the saturation limit of the detection system. A similar issue is encountered in absorption spectroscopy, except in absorption spectroscopy one is limited to quantifying an exceedingly small transmission through the gas cell. In SPS spectroscopy, reaching the saturation limit of the polarimeter can be avoided by reducing the applied electric field. In absorption spectroscopy, a different optical transition of the molecule is targeted where transmission is higher. Given the potential limited wavelength tunability of the laser source used in an absorption spectrometer, it may not be possible to target a different transition with the same laser. For the SPS spectrometer the same optical transition can be targeted by varying the applied electric field without changing the laser source. As a result, by adjusting the magnitude of the electric field an SPS spectrometer can obtain a dynamic range of concentration measurement that is larger than can be obtained by absorption spectroscopy. The extended dynamic range of concentration measurement results in an SPS based sensor that can monitor VHP concentrations over the entire decontamination cycle.

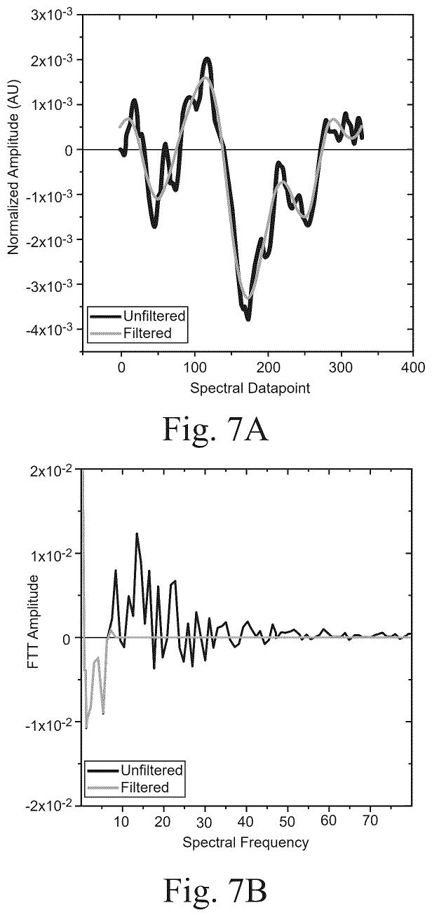

The attribute of immunity to parasitic etalon fringes of an SPS sensor that uses an modulated electric field may be violated by the following technical complications if: 1) there is electromagnetic pick-up in the laser current supply associated with driving the high-voltage electrode, and/or 2) there is electromagnetically-induced vibration of the high voltage electrode that results in intensity modulation of the laser beam that passes between the metal plates. Electromagnetic pick-up between the laser current supply and the high-voltage circuit can be resolved by appropriately shielding the laser current supply in a Faraday cage and increasing the physical distance between the high-voltage circuit. The impact of electromagnetically-induced vibration can be reduced by increasing the separation between the electrodes in the cell, or by careful design of the gas cell and high-voltage electrode so that the electric field modulation frequency does not overlap with the mechanical resonances of the system. In addition to sensor design modifications, the influence of parasitic etalon fringes in the spectral data can be mitigated by removing the periodic frequencies associated with the etalon fringes in the acquired spectral data. This approach is possible by using Fast Fourier Transform (FFT) filtering and removing the frequencies associated with etalon fringes observed in the spectral domain. An example of FFT filtering of Stark Polarization Spectrum data is provided in FIGS. 7A-7B and demonstrates the efficacy of this approach to mitigate the impact of parasitic etalon fringes without a physical re-design of the sensor. FIGS. 7A-7B show unfiltered and filtered Stark effect spectra for VHP (FIG. 7A) acquired using a near-infrared laser diode (1.4 μm) and a plot of the spectral amplitudes in frequency space of the spectra (FIG. 7B) following application of a Fast Fourier Transform to the spectral data shown in FIG. 7A. The gas cell temperature and pressure were 45° C. and 120 Torr respectively, and the peak applied electric field to the sample was 7000 V/cm at a modulation frequency of 11.65 kHz. A VHP concentration of 600+/−50 ppbv was measured upstream of the gas cell using an electrochemical sensor.

A preferred gas sensor based on Stark polarization spectroscopy has a gas cell with a pair of electrodes between which a modulated electric field is generated. A wavelength tunable laser source is included to measure optical transitions of VHP and water. Polarization optic(s) are included if the laser does not possess a known, unvarying, single polarization state output to set the polarization state of the light that is used to probe the electric field induced linear dichroism and birefringence in the vicinity of the VHP transitions. The polarization state of the light can be linearly or elliptically-polarized. In the embodiment described here, linearly-polarized light with a plane of polarization that is known with respect to the electric field vector is utilized to probe the VHP optical transitions. A voltage amplifier is connected to one of the electrodes in the gas cell. A polarimeter is comprised of a polarizer and a photodetection unit. The polarimeter is used to measure the induced linear dichroism and birefringence in the vicinity of optical transitions belonging to VHP and water. A lock-in amplifier is used to demodulate the change in the laser light polarization at the reference modulation frequency. The output reference from the lock-in amplifier can be used to modulate the electric field and/or the laser wavelength. An ADC or computer interface is used to record the demodulated voltage from the lock-in amplifier.

The polarimeter can be of type 90° (nearly-crossed), 45° (balanced), or hybrid. The reference output from the lock-in amplifier or a waveform generator can be sent to the voltage amplifier and/or a current modulation input on the laser driver. If the electric field and the laser wavelength are modulated, a lock-in amplifier capable of dual demodulation, or two lock-in amplifiers in series must be used to demodulate the voltage output from the polarimeter. The modulation frequency of the electric field may be smaller than the modulation frequency of the wavelength due to technical limitations in the current output of the voltage amplifier.

The modulated output of the polarimeter is recorded and averaged typically without demodulation, although this is not required. This corresponds to the direct absorption signal recorded in the experiment.

The relationship between the VHP concentration and the observed Stark polarization spectrum can be calibrated by exposing the system to known gas phase concentrations of VHP at fixed electric field amplitudes.

Due to differences in the response of VHP and water vapor to the applied electric field on the basis of molecular polarizability, it is possible for the system described to measure ppbv concentrations of VHP in the presence of parts per thousand by volume levels of water vapor without optical interference.

Due to the direct relationship between the Stark polarization signal and the magnitude of the electric field, it is possible to avoid saturation or increase detection sensitivity of the polarimeter by reducing or increasing the electric field magnitude. This allows the system described to use the same VHP optical transitions for concentration measurements spanning the ppbv to 1000 ppmv range. The concentration of water vapor generating a Stark polarization signal can be determined by analysis of the direct absorption spectrum for a fixed electric field amplitude. The relationship between water vapor concentration and the Stark polarization signal described can be used for in-line calibration of the system described while measuring gas effluent from the pharmaceutical manufacturing equipment decontamination process. Changes in Stark polarization response versus water concentration can be used to scale the VHP concentration estimate from the VHP Stark Polarization system. This calibration methodology can help account for changes in laser optical power and polarizer angle of a fielded instrument.

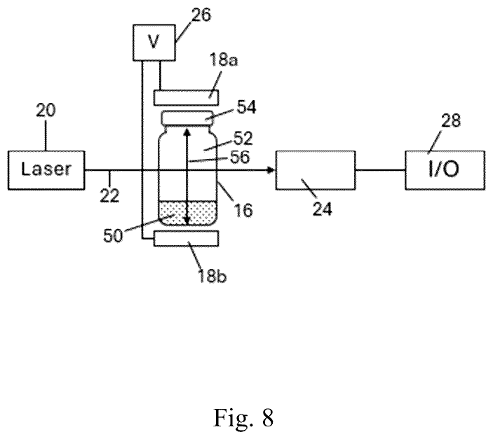

FIG. 8 depicts a system and method where a sealed vial 16 (e.g., glass or plastic) is positioned between two electrodes, 18a and 18b. In the bottom of the sealed vial is an active pharmaceutical product 50. The vial can be backfilled with an inert gas such as N₂ or Ar in headspace 52 and sterility can be maintained by sealing the top of the vial with an elastomer stopper 54 that is held in place using a metal crimp cap.

A light source 20 (with or without a polarizer) is oriented to pass a beam 22 of a known polarization state through the headspace 52 of the glass vial. A laser source may be used. The propagation direction of the beam 22 is perpendicular to the electric field generated between electrodes 18a and 18b in headspace 52. The polarized light may be linear (plane polarized) or elliptically-polarized, as circular and linearly polarized light will interact with the Stark-effect induced linear dichroism and birefringence associated with optical transitions of any H₂O, CO₂, or O₂ gases that have leaked into the headspace of the vial from the ambient environment due to a compromise of the seal.

A voltage source 26 supplies a voltage across electrodes 18a and 18b (one of which may be held at ground or reference voltage) to create an electric field directed along the long axis 56 of the vial. The electric field is isolated to the headspace of the vial such that a SP signal is only generated by any H₂O, CO₂, and O₂ that are present in the headspace of the vial where the electric field is located. H₂O, CO₂, and O₂ gas located outside the vial headspace where the electric field is absent will not be measured using SPS.

A detection subsystem 24, including for example, signal processing, computational, computer, and/or other subsystems is configured to detect the change in the polarization state of the laser beam after it passes through the head space of the glass vial where H₂O, CO₂, and O₂ may be present, and based on the detected change in the polarization state of the laser beam, to determine the concentrations of H₂O, CO₂, and O₂. The change in polarization state is a function of the concentration of the gas in the gas cell. The results can be provided to the user or other electronic systems via I/O section 28.

In still other examples, container 16 including headspace 52 is analyzed to ascertain information about the gases in the headspace (e.g., security applications, fermentation, and the like). Again, container 16 is positioned between two electrodes, 18a and 18b. In the bottom of the container is typically a product of the same type.

A light source subsystem 20 (e.g., a laser source) (with or without a polarizer) is oriented to pass a light beam 22 of a known polarization state through the headspace 52 of the container. The propagation direction of the light beam 22 is perpendicular to the electric field generated between electrodes 18a and 18b in headspace 52. The polarized light may be linear (plane polarized) or elliptically-polarized, as circular and linearly polarized light will interact with the Stark-effect induced linear dichroism and birefringence associated with optical transitions a gas or gases that have present in the head space of the container.

A voltage source 26 supplies a voltage across electrodes 18a and 18b (one of which may be held at ground or reference voltage) to create an electric field preferably directed along the long axis 56 of the container. The electric field is isolated to the head space of the container such that a SP signal is only generated by a gas or gases that are present in the headspace of the container where the electric field is located. Gases located outside the container headspace where the electric field is absent will not be measured using SPS.

A detection subsystem 24, including for example, signal processing, computational, computer, and/or other subsystems is configured to detect the change in the polarization state of the laser beam after it passes through the head space of the container, and based on the detected change in the polarization state of the laser beam, to determine the concentrations of a gas or gases. The change in polarization state is a function of the concentration of the gas in the container.

The results can be provided to the user or other electronic systems via I/O section 28 to detect, for example, explosive materials present in the container (in security applications), the level of fermentation taken place, or the like.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A method of monitoring the concentration of hydrogen peroxide gas, the method comprising:
driving gas sampled from a system that includes hydrogen peroxide gas and water vapor to a gas cell;
passing a light beam at a known polarization state through the gas cell and the hydrogen peroxide gas and water vapor therein;
creating an electrical field in the gas cell to reduce optical interference from water vapor while preserving the ability to more accurately measure the concentration of hydrogen peroxide gas in the gas cell;
detecting a change in the polarization state of the light beam after it passes through the hydrogen peroxide gas and water vapor due to the quadratic stark effect; and
based on the detected change in the polarization state of the light beam, determining the concentration of the hydrogen peroxide gas in the gas cell.

2. The method of claim 1 in which the system includes an enclosure and gas is sampled from the enclosure following decontamination.

3. The method of claim 1 further including modulating the strength of the electrical field and/or the light beam wavelength to improve detection of the hydrogen peroxide concentration.

4. The method of claim 1 including polarization optics between the light source and the gas cell to control the polarization state of the light.

5. The method of claim 4 in which the polarization optics are configured to pass a known polarization of light through the gas sample exposed to the electrical field in the gas cell.

6. The method of claim 1 including a polarimeter downstream of the gas cell for detecting the change in the polarization state of the light beam after it passes through the hydrogen peroxide gas and water vapor.

7. The method of claim 6 further including a calibration sequence which simultaneously measures changes in transmission and light beam polarization from water vapor used to calibrate the polarimeter.

8. The method of claim 6 in which the gas cell includes spaced electrodes connected across a voltage source for creating the electrical field in the gas cell and there is a polarimeter which receives the light beam after it exits the gas cell, the method further including delivering an output of the polarimeter to a data acquisition system to directly record the output, or to a phase sensitive detector which outputs a signal to the voltage source, light source, or both.

9. The method of claim 1 in which the light source is a visible, near infrared or infrared laser.

10. The method of claim 2 further including:
filling the enclosure with hydrogen peroxide gas to decontaminate the enclosure, driving gas including hydrogen peroxide gas and water vapor from the enclosure to a gas cell, passing a polarized light beam from a light source at a known polarization state through the gas cell and the hydrogen peroxide gas and water vapor therein, creating an electrical field in the gas cell to reduce interference of the water vapor while optically measuring the concentration of the hydrogen peroxide gas in the gas cell, detecting a change in the polarization state of the light beam after it passes through the hydrogen peroxide gas and water vapor, and based on the detected change in the polarization state of the light beam, determining the concentration of the hydrogen peroxide gas in the gas cell and barrier isolator; and
purging the enclosure to remove hydrogen peroxide gas therefrom, driving enclosure gas including hydrogen peroxide gas and water vapor to a gas cell, passing a laser beam from a light source at a known polarization state through the gas cell and the hydrogen peroxide gas and water vapor therein, creating an electrical field in the gas cell to reduce any interference of the water vapor in optically measuring the concentration of the hydrogen peroxide gas in the gas cell, detecting a change in the polarization state of the light beam after it passes through the hydrogen peroxide gas and water vapor, and based on the detected change in the polarization state of the light beam, determining the concentration of the hydrogen peroxide gas in the gas cell and enclosure.

11. A method of monitoring the level of an individual gas, the method comprising:
passing a light beam from a light source at a known polarization state through the gas and vapor in a container;
creating an electrical field in the container to reduce optical interference from other gases while preserving the ability to more accurately measure the concentration of the individual gas;
detecting a change in the polarization state of the light beam after it passes through the gas and vapor due to the quadratic stark effect; and
based on the detected change in the polarization state of the light beam, determining the concentration of the gas.

12. The method of claim 11 in which the gas is a decontamination gas.

13. The method of claim 12 in which the decontamination gas is comprised of hydrogen peroxide and water vapor.

14. The method of claim 11 further including modulating the strength of the electrical field and/or the laser beam wavelength to improve determination of the gas concentration.

15. The method of claim 11 further including applying Fast Fourier Transform filtering during detecting a change in the polarization state of the laser beam to remove optical interferences from parasitic etalon fringes not associated with polarization changes from gases.

16. The method of claim 11 including using a polarization optics to set the polarization state of the laser beam.

17. The method of claim 16 in which the polarizer is configured to polarize the laser beam at an angle of 45 degrees with respect to the orientation of the electrical field.

18. The method of claim 11 including using a polarimeter for detecting the change in the polarization state of the laser beam after it passes through the gas.

19. The method of claim 11 in which the light source is a visible, near infrared or infrared laser.

20. A method of monitoring the concentration of gases in the headspace of a container, the method comprising:
passing a light beam from a light source at a known polarization state through the container headspace;
applying an electric field through the headspace of the container such that only optical signals from gases in the headspace of the container are measured while removing the interference from optical signals generated by those gases present in an environment outside the container;
detecting a change in the polarization state of the light beam after it passes through the headspace region of the container; and
based on the detected change in the polarization state of the light beam, determining the concentration of individual gases in the headspace of the container.

21. The method of claim 20 in which the container includes a pharmaceutical product and the headspace may include gases that have leaked into the container.

22. The method in claim 21 in which the gas that has leaked into the container may include O₂, N₂, H₂O, and CO₂.

23. The method of claim 20 further including modulating the strength of the electrical field and/or the light beam wavelength to improve the detection concentration range of the gases located inside the container.

24. The method of claim 21 further including polarization optics between the light source and the container to control the polarization state of the light beam.

25. The method of claim 20 including a polarimeter downstream of the container for detecting the change in the polarization state of the light beam.

26. The method of claim 20 in which the light source is a visible, near infrared, or infrared laser.
